(19) United States
(12) Plant Patent Application Publication
(10) Pub. No.: US 2004/0034891 P1
(43) Pub. Date: Feb. 19, 2004

(54) Apple tree named 'Dalitoga'

(76) Inventor: Guy Raymond Ligonniere, Angers (FR)

Correspondence Address: STRATTON BALLEW, 213 S 12TH AVE, YAKIMA, WA, 98902, US

(21) Appl. No.: 10/639,179

(22) Filed: Aug. 11, 2003

(30) Foreign Application Priority Data
Aug. 13, 2002 (QZ) 2002/1259

(51) Int. Cl. A01H 5/00

(52) U.S. Cl. PLT/162

(57) ABSTRACT

A new and distinct Gala-type apple cultivar is disclosed. The new cultivar ‘Dalitoga’ arose as a whole tree mutation of ‘Imperial Gala.’ ‘Dalitoga’ is notable for its intense coloration and early maturity as compared to its parent and to other known Gala cultivars.

### CROSS REFERENCE TO RELATED APPLICATIONS

None

### PRIORITY CLAIM

This application claims priority under 35 U.S.C. §1 19(f) of European Plant Variety Rights application Ser. No. 2002/1259 filed Aug. 13, 2002.

### STATEMENT REGARDING FEDERALLY SPONSORED RESEARCH OR DEVELOPMENT

None

### LATIN NAME OF THE GENUS AND SPECIES OF THE PLANT CLAIMED

Malus Mill.

### VARIETY DENOMINATION

‘Dalitoga’

### BRIEF DESCRIPTION OF THE PHOTOGRAPHS

FIG. 1 shows the fruit and leaves of ‘Dalitoga.’

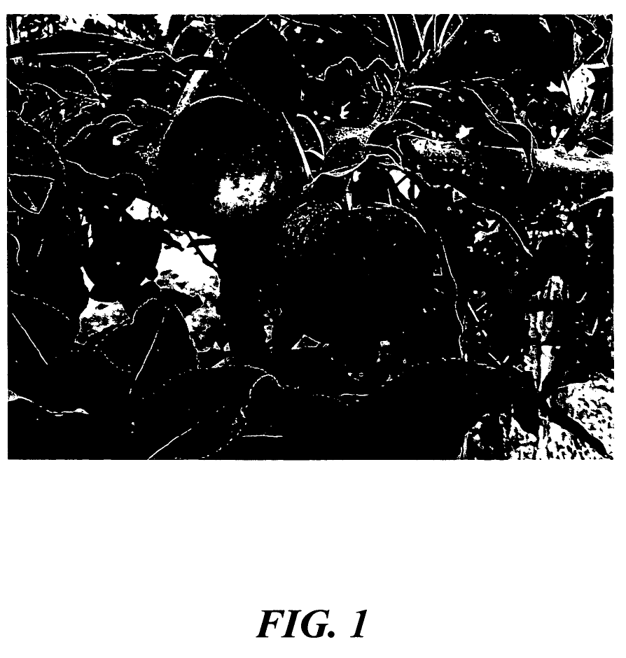

FIG. 2 shows the fruit, leaves and trunk of ‘Dalitoga’

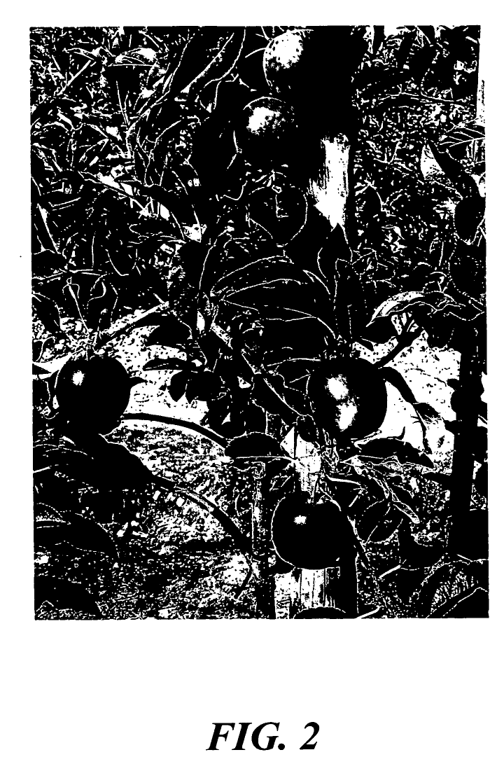

FIG. 3 shows the ‘Dalitoga’ tree.

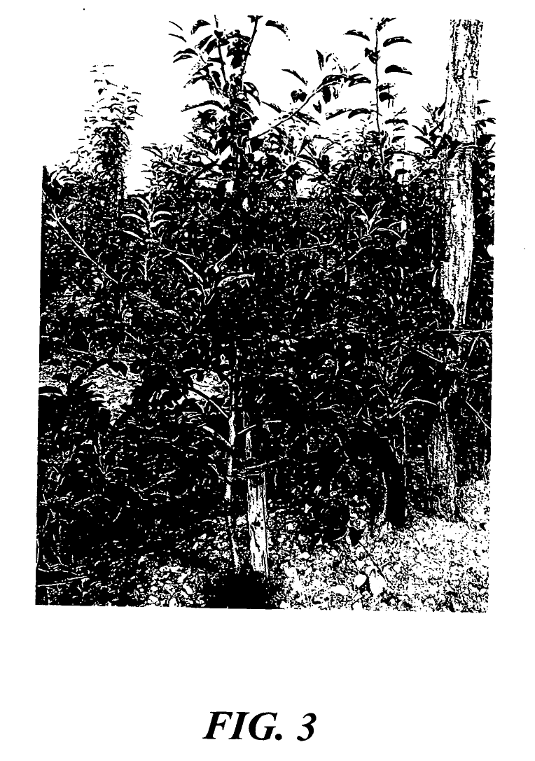

FIG. 4 shows a branch and leaves (1 year old shoot) of ‘Dalitoga.’

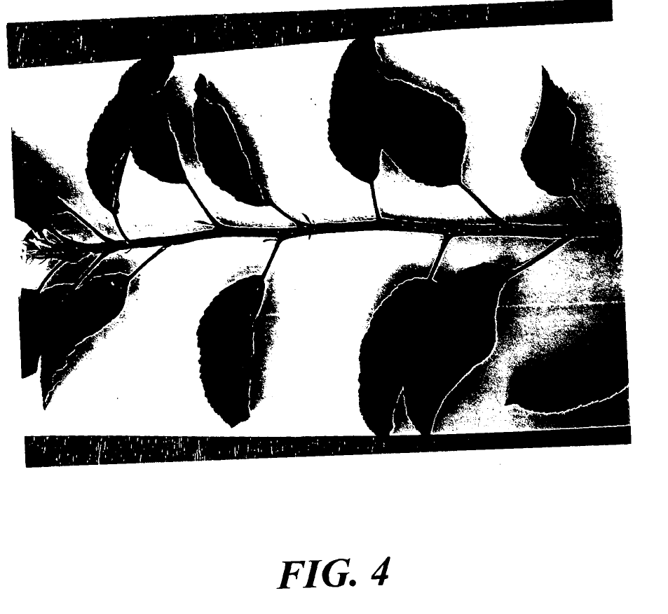

FIG. 5 shows the flesh of fruit of ‘Dalitoga.’

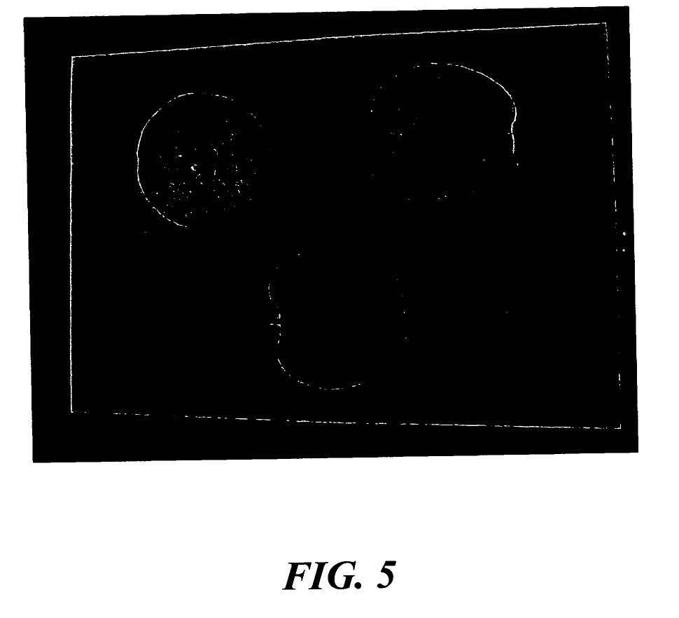

FIG. 6 shows ‘Dalitoga’ as compared with ‘Galaxy’ Gala (U.S. Plant Pat. No. 6,955) and Brookfield® ‘Baigent’ Gala (U.S. Plant Pat. No. 10,016).

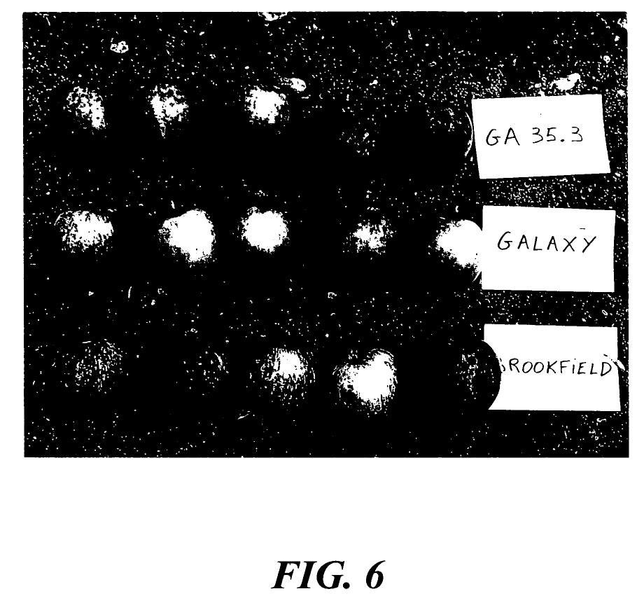

FIG. 7 shows the difference in maturity time between ‘Dalitoga’ and ‘Galaxy’ Gala with a comparison between the starch levels, foreground and background colors of the two varieties picked on the same day.

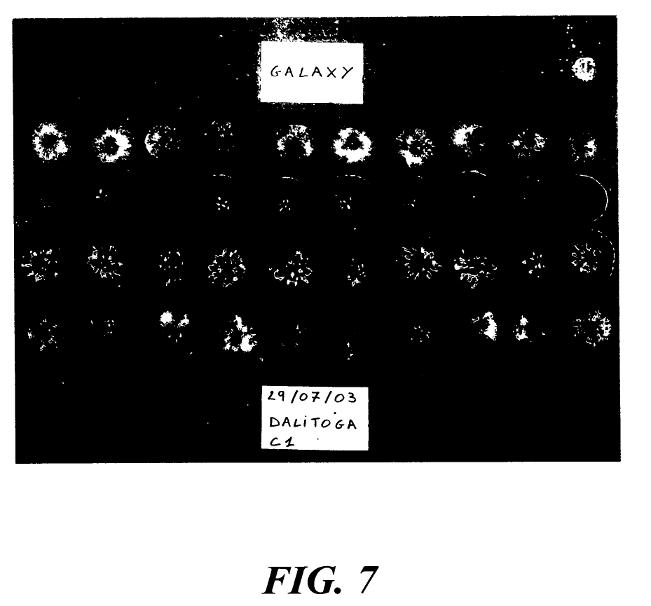

### BRIEF DESCRIPTION OF THE VARIETY

‘Dalitoga’ is a whole tree mutation of ‘Imperial Gala’ (not patented). The original mutation was discovered in 1998 in a cultivated block of ‘Imperial Gala’ trees near Lezigne, France, and was asexually propagated for testing purposes at Doue La Fontaine, France in 1999 under breeder's reference number R25 A35-GA 35.3. Asexually propagated progeny of ‘Dalitoga’ have been found to retain the unique characteristics of the parent, and remain true to type through successive generations.

‘Dalitoga’ is similar to its parent in terms of tree shape, vigor, and productivity. However, ‘Dalitoga’ is distinguishable from its parent in the following respects:

| Characteristic | Imperial Gala | ‘Dalitoga’ |
|---|---|---|
| Date of full bloom | Typical for Gala | 1-3 days earlier |
| | (compare to ‘Galaxy’) | |
| Rate of fruit growth | Typical for Gala | 4 mm-8 mm larger than |
| | (compare to ‘Galaxy’) | 'Imperial Gala’by mid-July |
| Coloration | 30% to 50% colored | 50% to 95% colored |
| | | (avg. 75%) |
| Maturity | Last week of August | First week of August |

### DETAILED BOTANICAL DESCRIPTION OF THE VARIETY

The following is a detailed botanical description of ‘Dalitoga,’ a new and distinct cultivar of Malus Mill., based on observations made on the original tree from 1998 to 2001, and during the 2002 and 2003 growing seasons of specimens of the second generation planted at Angers, France in 2001 and 2002. All colors are described according to The Royal Horticultural Society Color Chart n°222879/2001. It should be understood that the botanical and analytical characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.

1. Tree:

Vigor.—Medium.

Type.—Ramified.

Habit.—Spreading.

2. Dormant one year old shoot:

Pubescence.—Weak.

Thickness.—Medium (48 mm).

Internode length.—Long (avg. 26 mm).

Number of lenticels.—Medium (avg. 9/cm²).

3. Flowers:

Color (balloon stage).—Red Purple from 73A to 74B.

Diameter of open flower.—Medium (avg. 38 mm).

Relative position of petal margins.—Slightly overlapping.

4. Leaf:

Attitude in relation to shoot.—Outward (horizontal).

5. Leaf blade:

Length.—Medium (avg. 89 mm).

Width.—Medium (avg. 47 mm).

Length-width ratio.—Medium.

Margin.—Serrate.

6. Petiole:

Length.—Medium to long (avg. 27 mm).

7. Fruit:

Size.—Medium (avg. 67.5 mm).

Ratio of height to width.—Medium to large.

General shape in profile.—Oblong to oblong conical.

Position of maximum diameter.—Middle.

Ribbing.—Weak.

Crowning at calyx end.—Weak.

Aperture of eye.—Closed.

Size of eye.—Small (avg. 9.4 mm).

Length of sepal.—Medium (avg. 7.9 mm).

Depth of eye basin.—Medium (avg. 7.9 mm).

Width of eye basin.—Medium (avg. 27.4 mm).

Thickness of stalk.—Medium (avg. 3.7 mm).

Length of stalk.—Short to medium (avg. 16.3 mm).

Depth of stalk cavity.—Medium to deep (avg. 11.8 mm).

Width of stalk cavity.—Medium (avg. 31.5 mm).

Size of lenticels.—Small (avg. 0.7 mm).

Bloom of skin.—Weak.

Greasiness of skin.—Very weak.

Ground color of skin.—Yellow-Green 154C to Yellow 4C.

Over color of skin.—Red 46B to Red Orange 34A.

Amount of over color.—High.

Intensity of over color.—Medium.

Pattern of over color.—50% to 95% stripes (avg. 75%) with solid flush in some parts.

Quantitative analysis of fruit:

| | DALITOGA (First picking, 29 July 2003 | | | |
|---|---|---|---|---|
| | | | | Starch |
| Fruits | Pressure 1 | Pressure 2 | Sugar | 0 to 10 |
| 1 | 8.6 | 8.1 | 12.8 | 4 |
| 2 | 8.8 | 8.7 | 11.8 | 5 |
| 3 | 9.8 | 9.5 | 12.6 | 3 |
| 4 | 8.5 | 7.2 | 14 | 6 |
| 5 | 9.7 | 8 | 12.4 | 5 |
| 6 | 8.1 | 9.2 | 12.6 | 3 |
| 7 | 7.8 | 7.4 | 14.2 | 5 |
| 8 | 7.8 | 6.6 | 13 | 7 |
| 9 | 10.3 | 10.1 | 12 | 3 |
| 10 | 7.8 | 7.7 | 13.6 | 5 |
| Avg. | 8.72 | 8.25 | 12.9 | 4.6 |

Aroma.—Slightly more intense than ‘Imperial Gala’.

Use.—Fresh market.

Resistance to known diseases.—None noted.

Storageability.—Good; in limited early trials, fruit retained firmness, taste and texture after three months in real atmosphere storage.

We claim:

1. A new and distinct apple tree, substantially as shown and described herein.
